(19) United States
(12) Patent Application Publication
(10) Pub. No.: US 2015/0359827 A1
(43) Pub. Date: Dec. 17, 2015

(54) BLUE-GREEN ALGAE EXTRACT MIXTURES AND METHODS OF USE

(71) Applicant: Cerule, LLC, Klamath Falls, OR (US)

(72) Inventors: Gitte S. Jensen, Klamath Falls, OR (US); Jesse Guthrie, Klamath Falls, OR (US)

(73) Assignee: CERULE, LLC, Klamath Falls, OR (US)

(21) Appl. No.: 14/731,108

(22) Filed: Jun. 4, 2015

Related U.S. Application Data
(60) Provisional application No. 62/012,174, filed on Jun. 13, 2014.

(51) Int. Cl. A61K 35/748

(52) U.S. Cl. CPC A61K 35/748

(57) ABSTRACT

Disclosed herein are compositions including a mixture of an aqueous extract of Aphanizomenon flos aquae and an aqueous extract of Arthrospira. The compositions are of use for inducing stem cell trafficking (such as stem cell mobilization and homing) in a subject. Thus, methods for increasing stem cell trafficking that include administering a effective amount of the mixture of the aqueous extract of Aphanizomenon fibs aquae and the aqueous extract of Arthrospira, are disclosed herein.

### CROSS-REFERENCE TO RELATED APPLICATION

This application claims priority to U.S. Provisional application No. 62/012,174, filed on Jun. 13, 2014, which is incorporated by reference herein in its entirety.

### FIELD

This application relates to mixtures of aqueous extracts from blue-green algae and their use to increase stem cell trafficking in a subject.

### BACKGROUND

Stem cells are pluripotent or multipotent cells derived from somatic tissue capable of differentiating into more specialized cells. Stem cells have been found to differentiate into a variety of tissue-specific cell types, such as myocytes, hepatocytes, osteocytes, glial cells, and neurons, where they play an important role in the healing and regenerative processes of various tissues and organs. For example, hematopoietic stem cells originate in bone marrow and can differentiate into many different types of blood cells, including red blood cells, platelets, and leukocytes. Endothelial stem cells (ESCs) also originate in bone marrow and can mobilize to the circulatory system and home to target tissue and differentiate into endothelial cells, which line the inner layer of blood vessels.

Many studies suggest that the trafficking of stem cells from bone marrow to target tissue constitutes a natural phenomenon of healing in the human body. For example, stem cells can follow concentration gradients of cytokines released by damaged tissues and migrate on their own into tissues following such gradients. Therefore, activation and enhancement of stem cell trafficking can amplify these physiological processes and provide a potential therapy for various pathologies.

Prior work has shown that some extracts of blue green algae can be used to stimulate mobilization of certain types of stem cells. However, there remains a need for improved formulations of blue-green algae to increase stem cell trafficking, such as increased stem cell mobilization and homing to target tissue.

### SUMMARY

Disclosed herein is the unexpected finding that a composition including a mixture of an aqueous extract of AFA and an aqueous extract of Arthrospira acts synergistically to increase stem cell trafficking in human subjects.

Several embodiments provide a composition comprising a first component and a second component, wherein the first component comprises an aqueous extract of Arthrospira, and the second component comprises an aqueous extract of AFA. In some embodiments, the extracts can be of fresh, dehydrated, or preserved Arthrospira or AFA. The aqueous extract can be, for example, a water extract. In some embodiments, the Arthrospira comprises Arthrospira platensis, Arthrospira maxima, or both. In some embodiments, the first component and the second component can be mixed at a ratio of about 10:90 to about 90:10 w/w (such as about 10:90, about 20:80, about 30:70, about 40:60, about 50:50, about 60:40, about 70:30, about 80:20, or about 90:10 w/w).

The composition can be formulated for administration to a subject. In some embodiments, a composition including the extract mixture alone, or including other extracts of AFA or Arthrospira can be administered to a subject in need of stem cell mobilization and/or increased number of circulating stem cells.

A method is disclosed herein for increasing stem cell trafficking by administering a therapeutically effective amount of a mixture of an aqueous extract of AFA and an aqueous extract of Arthrospira to a subject. The extracts can be administered alone or in conjunction with other agents, such as other extracts of blue-green algae. The administration of the therapeutically effective amount of the mixture of the aqueous extract of AFA and the aqueous extract of Arthrospira can induce an increase in the number of certain stem cells, such as CD34+ stem cells and/or CD45−CD31+KDR+ stem cells, in the subject's circulatory system.

The foregoing and other features and advantages will become more apparent from the following detailed description of several embodiments, which proceeds with reference to the accompanying figures.

### BRIEF DESCRIPTION OF THE FIGURES

FIG. 1 is a graph illustrating the expression of CD25 on the surface of natural killer T cells (NKT cells) following incubation with interleukin-2 (IL-2; positive control), an aqueous extract of AFA (AFA-w), an aqueous extract of Arthrospira (SPIR-w), and baseline control.

FIG. 2 is a graph illustrating Granulocyte-macrophage colony-stimulating factor (GM-CSF) expression on the surface of CD3+ peripheral blood mononuclear cells (PBMCs) following incubation with IL-2 (positive control), AFA-w, SPIR-w, or baseline control.

FIG. 3 is a graph illustrating GM-CSF expression on the surface of CD14−/CD3− PBMCs following incubation with IL-2, AFA-w, SPIR-w, or control (baseline).

FIG. 4 is a schematic diagram illustrating how signaling via L-selectin induces CXCR4 expression. The chemokine receptor CXCR4 is stored in intracellular reserves. When an L-selectin ligand binds to L-selectin, CXCR4 is rapidly moved to the cell surface, which makes the cell more sensitive to chemotactic signals from tissue. This can increase recruitment of such cells from the blood into the tissue.

FIG. 5 is a graph illustrating the effects of AFA-w, SPIR-w, and a 50:50 mixture of AFA-w and SPIR-w (“Blend”) on cell-surface CXCR4 expression in CD34+ KG1a cells. The KG1a cell line is a stem cell-like CD34+ progenitor cell line known to express L-selectin, and to undergo induction of CXCR4 expression upon ligand binding to L-selectin. Surprisingly, although the Blend contained half the amount of AFA-w and SPIR-w as either of the extract alone conditions, an increase in CXCR4 expression was observed in the Blend condition compared to treatment with either of the extracts alone.

FIG. 6 is a schematic diagram of the protocol used to study the effect of AFA-w, SPIR-w, and a mixture thereof, on stem mobilization in humans.

FIG. 7 is a graph illustrating mobilization of the CD45dim CD34+ KDR− stem cell subset in human subjects after consumption of AFA-w, SPIR-w, and a 50:50 mixture thereof (“Blend”). The percent change compared to baseline stem cell numbers in the blood circulation was calculated for the four study participants, and then averaged.

FIG. 8 is a graph illustrating mobilization of the CD45−CD31+KDR+ stem cell subset in human subjects after consumption of AFA-w, SPIR-w, and a 50:50 mixture thereof (“Blend”). CD45−CD31+KDR+ stem cells are endothelial stem cells that originate in bone marrow and traffic to endothelial tissue. The percent change compared to baseline stem cell numbers in the blood circulation was calculated for the four study participants, and then averaged. Surprisingly, although the Blend contained half the amount of SPIR-w as the SPIR-w assay, a comparable increase in CD45−CD31+KDR+ stem cells was identified at the two hour post-administration time point.

FIG. 9 is a graph illustrating mobilization of the CD34+ very small stem cell subset in human subjects after consumption of AFA-w, SPIR-w, and a 50:50 mixture thereof (“Blend”). The percent change compared to baseline stem cell numbers in the blood circulation was calculated for the four study participants, and then averaged.

### DETAILED DESCRIPTION

### I. Summary of Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references. To facilitate review of the various embodiments, the following explanations of terms are provided:

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes. If administered orally, the AFA and Arthrospira extracts may be provided or administered in the form of a unit dose in solid, semi-solid, or liquid dosage form such as tablets, pills, powders, liquid solutions, or liquid suspensions. However, extracts of blue-green algae also may be administered intravenously in any conventional medium for intravenous injection, such as an aqueous saline medium, or in a blood plasma medium.

Agent that increases stem cell circulation: A compound or composition (such as a mixture of aqueous extracts of AFA and Arthrospira) that increases the release of stem cells from bone marrow into the circulatory system. The agent may increase the circulation of certain types of stem cells, such as CD45−CD31+KDR+ stem cells and/or CD34+ stem cells.

Animal: A living, multicellular, vertebrate organism including, for example, mammals, fish, reptiles, and birds.

Biological sample: A biological specimen containing cells, DNA, RNA, protein, or combinations thereof, obtained from a subject. Examples include, but are not limited to, saliva, blood, plasma, serum, urine, tissue, hair, cells, tissue biopsy, surgical specimen, fecal matter, and autopsy material.

Blue-green algae: Common name for gram-negative photosynthetic bacteria belonging to Division Cyanophyta that may exist in unicellular, colonial, or filamentous forms. Representative blue-green algae include, but are not limited to, Arthrospira species and Aphanizomenon species. AFA is one specific, non-limiting type of blue-green algae.

The term “algae” is the plural form of “alga,” which is a cell of a microalgae species. For example (and without limitation), “blue-green algae” refers to multiple cells of a single Aphanizomenon species, multiple cells of a single Arthrospira species, or a mixture of cells from multiple Aphanizomenon and/or Arthrospira species.

Circulatory system: In animals, the circulatory system is composed of the structures that move blood and blood components throughout the body, including the vascular and lymph systems. The components of the circulatory system include the heart, blood vessels (arteries, veins, and capillaries), and lymph vessels.

Circulating stem cell: A stem cell present in the circulatory system.

Component of blue-green algae: Any fraction, extract, or isolated or purified molecule from a blue-green algae cell. In one embodiment, the component is an aqueous extract of a blue-green algae, such as AFA or Arthrospira.

Control: A “control” refers to a sample or standard used for comparison with a test sample, such as a tissue sample obtained from a healthy subject (or plurality of subjects). In some embodiments, the control is a sample obtained from a healthy subject (or plurality of subjects) (also referred to herein as a “normal” control). In some embodiments, the control is a historical control or standard value (i.e. a previously tested control sample or group of samples that represent baseline or normal values, such as baseline or normal values in a healthy subject). In some examples the control is a standard value representing the average value (or average range of values) obtained from a plurality of patient samples (such as an average value or range of values of circulating stem cells, from normal patients).

Endothelial cell: A cell from the endothelium, which is the thin layer of cells that line the interior surface of blood vessels.

Extract: A concentrated preparation of a composition, such as a blue-green algae, obtained by removing active constituents of the composition with suitable solvents (such as water), evaporating the solvent, and adjusting the residual mass or powder to the a pre-determined standard amount. An aqueous extract is a water-containing extract, and in some examples may be pure water. An aqueous extract initially obtained by solvent extraction or may be converted to a dried form and still considered an extract.

Hematopoiesis: The formation and development of blood cells. Hematopoiesis involves the proliferation and terminal differentiation of hematopoietic stem cells. In adult mammals, hematopoiesis is known to occur in bone marrow. Hematopoiesis is the production of hematopoietic cells including B cells, T cells, cells of the monocyte macrophage lineage, and red blood cells.

Increase: A significant increase in a particular activity or of a component of interest, such as an increase in a particular parameter of a cell or organism. In one embodiment, an increase refers to a 25%, 50%, 100% or greater than 100% increase in a parameter. In one specific, non-limiting example, an increase in stem cell circulation refers to an increase in a specific population of the cells, such as a 25%, 50%, 100%, 200%, 400%, 500%, or greater increase in the specific population of cells or the response of the population of cells. In one embodiment, the parameter is the mobilization of stem cells. In another embodiment, the parameter is the differentiation of stem cells. In yet another embodiment, the parameter is the homing of stem cells.

Isolated: An “isolated” biological component (such as a nucleic acid molecule, polypeptide, polysaccharide, or other biological molecule) has been substantially separated or purified away from other biological components of cells in which the component naturally occurs. An “isolated” cell has been substantially separated or purified away from other cells of different species (in the case of microorganisms) or cells of the organism (in the case of multi-cellular organisms). Nucleic acids and proteins may be isolated by standard purification methods, recombinant expression in a host cell, or chemically synthesized. Cells may be isolated by standard culturing methods. In one embodiment, the blue-green algae is harvested from a natural source (such as Klamath Lake), and prepared by drying (see below).

L-selectin: A member of the selectin family of calcium-dependent lectins, also known as CD62L. An adhesion molecule used by stem cells to adhere to the bone marrow environment. L-selectin, the smallest of the vascular selectins, is a 74-100 kDa molecule, that is constitutively expressed at the tips of microfolds on granulocytes, monocytes, and a vast array of circulating lymphocytes, L-selectin is also known as LECAM-1, LAM-1, Mel-14 antigen, gp90^{mel}, and Leu8/TQ-1 antigen. L-selectin is known to be important for binding of leukocytes to endothelium in various physiological situations, including binding of phagocytes to endothelium, binding of leukocytes to inflamed endothelium, and lymphocyte homing and adhesion to high endothelial cells of post capillary venules of peripheral lymph nodes. Moreover, this adhesion molecule contributes greatly to the capture of circulating leukocytes during the early phases of the adhesion cascade. The amino acid sequences of many L-selectins are known.

An “L-selectin ligand” specifically binds L-selectin. In some embodiments, a ligand can block activation by other ligands, for example by spatial interference with the ligand binding area. A ligand can also activate the cell via ligation to L-selectin, for example by triggering calcium flux, cytoskeletal rearrangements, or other signaling events. In addition, a ligand can alter signal transduction pathways so a subsequent binding with either another L-selectin ligand, or an L-selectin-independent stimulus results in an altered physiological response. In some examples, when human lymphocytes are activated via some L-selectin ligands, L-selectin triggers the expression of CXCR4, a receptor for Stromal Derived Factor 1 (SDF1), a cytokine involved in the residence of stem cells in the bone marrow. In one embodiment, the L-selectin-containing extract of the blue-green algae inhibited the expression of CXCR4 triggered by the activation of L-selectin with Fucoidan. Amino acid sequences for exemplary L-selectin ligands are known. For example, Mus musculus GlyCam-1 is shown in GENBANK® Accession Number NM_{—}008134 and Human mRNA isolates for GlyCam-1 are shown in GENBANK® Accession Nos. AJ_{—}489 590, AJ 489 591, AJ 489 592, AJ 489 593, and AJ 489 589, all as available on Jun. 24, 2005, which are incorporated herein by reference. These amino acid sequences are not meant to be limiting, but are provided as examples. Recombinant and modified forms are included in the present disclosure.

Leukocytes: White blood cells. Spherical, colorless, and nucleated corpuscles involved in host defense, including immunological responses. Specific types of leukocytes include basophils, coelomocytes, eosinophils, haemocytes, lymphocytes, neutrophils, and monocytes, circulating dendritic cells, and circulating hematopoietic stem cells.

Mammal: This term includes both human and non-human mammals. Similarly, the term “subject” includes both human and veterinary subjects.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of mixtures of blue-green algae extracts described herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular embodiments, carrier may be sterile, and/or suspended in a unit dosage form containing one or more measured doses of a composition suitable to induce a desired response (such as an increase in circulating stem cells). It may also be accompanied by medications for its use for treatment purposes. The unit dosage form may be, for example, in a sealed vial that contains sterile contents or a syringe for injection into a subject.

Progenitor cell: A cell that gives rise to progeny in a defined cell lineage. A “hematopoietic progenitor cell” is a cell that gives rise to cells of the hematopoietic lineage.

Stem Cell: A pluripotent cell that gives rise to progeny of many tissue types, including (but not limited to) the entire hematopoietic and marrow stromal cell lineages. A typical stem cell resides in the bone marrow, either as an adherent stromal cell type, or as a more differentiated cell that expresses CD34, either on the cell surface or in a manner where the cell is negative for cell surface CD34. In some embodiments, a CD34 positive stem cell can be the size of a lymphocyte, or smaller.

In some embodiments, a stem cell can be a Very Small Embryonic-Like stem cell (VSEL), which is a primitive stem cell that can exhibit some pluripotent stem cell properties (e.g., capacity to differentiate into multiple tissue types) and expresses embryonic markers, such as CXCL4, SSEA-1, and Oct-4. VSELs are about 2-5 μM in diameter, which is smaller than an erythrocyte and larger than a platelet. (For review, see Zuba-Surma et al., Cytometry A, 75:4-13, 2009).

Another example of a type of stem cell is a CD45−CD31+KDR+ stem cell, which is an endothelial stem cell (ESC) that is found in bone marrow, and which can ultimately give rise to an endothelial cell that forms part of the thin-walled endothelium that lines the inner surface of blood vessels.

Alternatively, a stem cell can be a cell that can be measured by fluorescently labeled aminoacetaldehyde, formed when an enzyme in stem cell cytoplasm, converts a non-fluorescent substrate into a fluorescent compound that is retained inside the stem cell and allowing its detection based on enzymatic function.

Stem Cell Homing: The process of a stem cell migrating from the circulatory system into a tissue or organ. In some instances, homing is accomplished via tissue-specific adhesion molecules and adhesion processes. “Recruitment” of the stem cell from circulation to the target tissue can be facilitated by a compound or molecule, such as a chemoattractant signal or cell receptor.

Stem Cell Mobilization: The process of release of stem cells from the bone marrow or other tissue such as muscles, into circulating blood.

Stem Cell Trafficking: The processes of movement of a cell from the tissue of origin and traveling via the circulatory system to a target tissue. In one embodiment, trafficking includes movement of a cell from the tissue of origin, homing by adhesion to the endothelium, transmigration, and final migration within the target organ. In one embodiment, tracking is the process of movement of a cell of the immune system. In another embodiment, trafficking includes stem cell mobilization. One specific, non-limiting example of trafficking is the movement of a stem cell to a target organ.

Subject: An animal that has a circulatory system, including vertebrates such as humans and other veterinary subjects, such as, but not limited to, primates, canines, felines, bovines, and rodents.

Therapeutically effective amount: An amount of a pharmaceutical preparation that alone, or together with a pharmaceutically acceptable carrier or one or more additional therapeutic agents, induces the desired response, such as reducing or inhibiting one or more signs or symptoms associated with a condition or disease. Therapeutically effective amounts a therapeutic agent can be determined in many different ways, such as assaying for an increase in circulating stem cells. Therapeutically effective amounts also can be determined through various in vitro, in vivo or in situ assays.

Therapeutic agents can be administered in a single dose, or in several doses, for example daily, during a course of treatment. However, the effective amount of can be dependent on the source applied, the subject being treated, the severity and type of the condition being treated, and the manner of administration. When administered to a subject, a dosage will generally be used that will achieve target tissue concentrations.

In several embodiments, a therapeutically effective amount can be an amount of a composition including a mixture of an aqueous extract of AFA and an aqueous extract of Arthrospira, capable of triggering or increasing stem cell trafficking (such as mobilization and/or homing), which can be determined by various methods used in the biological sciences. These methods include, but are not limited to, generating an empirical dose-response curve. In one embodiment, a therapeutically effective amount is an amount effective for increasing mobilization of stem cells that replenish, repair, or rejuvenate tissue. In still another embodiment, the therapeutically effective amount is an amount effective for increasing homing of stem cells from the circulatory system to various tissues or organs.

In another embodiment, a therapeutically effective amount is an amount effective for increasing circulating stem cells (or a subpopulation of stem cells) in a subject. For example, a pharmaceutical preparation can increase the number of circulating stem cells in a subject by at least 20%, at least 30%, at least 40%, at least 50%, at least 70%, at least 90%, at least 98%, or even at least 100%, as compared to an amount in the absence of the pharmaceutical preparation.

Treating or Treatment: A therapeutic intervention that reduces a sign or symptom of a disease or pathological condition related to a disease. Treatment can also induce remission or cure of a condition.

Reducing a sign or symptom associated with a disease or condition can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular condition.

Under conditions sufficient for: A phrase that is used to describe any environment that permits a desired activity.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Compositions

Disclosed herein are non-naturally occurring mixtures of aqueous extracts of blue-green algae, such as a mixture of an aqueous extract of AFA with an aqueous extract of Arthrospira (such as Arthrospira platensis, Arthrospira maxima, or a combination thereof). The composition can be formed by mixing liquid or dried extracts, for example. If liquid extracts are mixed, the composite composition can then be dried and stored for future use. The extracts can be dried using standard processes (such as those described herein), and optionally can be re-suspended in an aqueous solution.

The extracts can be mixed at any ratio, such as a ratio of about 5:95, about 10:90, about 20:80, about 30:70, about 40:60, about 50:50, about 60:40, about 70:30, about 80:20, about 90:10, about 95:5 w/w of the aqueous extract of AFA to the aqueous extract of Arthrospira. As used herein with reference to a ratio of blue green algae extracts, “about” refers a tolerance of ±5%; for example, “about 50:50” includes a tolerance of ±2.5% for each aspect of the ratio (e.g., 47.5-52.5:47.5-52.5). In additional embodiments, the extracts can be mixed at a ratio of about 5:95 to about 95:5 w/w of the aqueous extract of AFA to the aqueous extract of Arthrospira, such as a ratio of about 10:90 to about 90:10, about 20:80 to about 80:20, about 30:70 to about 70:30, about 40:60 to about 60:40, about 45:55 to about 55:45, about 5:95 to about 10:90, about 10:90 to about 20:80, about 20:80 to about 30:70, about 30:70 to about 40:60, about 40:60 to about 50:50, about 50:50 to about 60:40, about 60:40 to about 70:30, about 70:30 to about 80:20, about 80:20 to about 90:10, or about 90:10 to about 95:5 w/w of the aqueous extract of AFA to the aqueous extract of Arthrospira.

In more embodiments, the extracts can be mixed at any ratio, such as a ratio of 1:99, 5:95, 10:90, 20:80, 30:70, 40:60, 45:55, 50:50, 55:45, 60:40, 70:30, 80:20, 90:10, 95:5, or 99:1 w/w of the aqueous extract of AFA to the aqueous extract of Arthrospira. In additional embodiments, the extracts can be mixed at a ratio of 1:99 to 99:1 w/w of the aqueous extract of AFA to the aqueous extract of Arthrospira, such as a ratio of 5:95 to 95:5, 10:90 to 90:10, 20:80 to 80:20, 30:70 to 70:30, 40:60 to 60:40, 45:55 to 55:45, 5:95 to 10:90, 10:90 to 20:80, 20:80 to 30:70, 30:70 to 40:60, 40:60 to 50:50, 50:50 to 60:40, 60:40 to 70:30, 70:30 to 80:20, 80:20 to 90:10, or 90:10 to 95:5 w/w of the aqueous extract of AFA to the aqueous extract of Arthrospira.

Blue-green algae, such as AFA or Arthrospira can be fractionated. Processes for growing, harvesting, and concentrating blue-green algae cells have been described. Blue-green algae, such as AFA or Arthrospira, can be isolated from any source. The source can be a natural source of blue-green algae, such as a lake (for example Klamath Lake). The source can also be a man-made source of blue-green algae such as an artificial lake or water source. The source can be an environment produced to grow and harvest blue-green algae commercially.

The blue-green algae can be used directly, or can be stored as liquid, frozen liquid, freeze-dried, or dried using known methods, such as those described below. In one embodiment, the blue-green algae are harvested and dried using REFRACTANCE WINDOW™ Technology. The term “REFRACTANCE WINDOW™ Technology” refers to a system wherein the dryer utilizes the very properties of water to drive water out of the product. In brief, when water is placed over a heating source, heat gets dispersed in the water through convection. As it absorbs heat, water transmits infrared energy to the outside in three ways: evaporation, conduction, and radiation. If the surface of the water surface is covered by a transparent medium such as plastic, evaporation and its associated heat loss are blocked and only conduction occurs. The plastic membrane acts like a minor reflecting infrared energy. When a moist material, such as wet blue-green algae is placed on the plastic surface, the water in the material creates a “window” that allows for the passage of infrared energy. It is believed that in this system the water in the material allows for radiation, conduction and evaporation all to occur, providing for exceptionally effective heat transfer. However after a few minutes, as the material dries, the infrared “window” closes and conduction remains the only means of heat transfer. Since plastic is a poor heat conductor, little heat is lost and transferred to the product. Therefore, when dried with REFRACTANCE WINDOW™ Technology, algae are exposed to heat only briefly.

In this drying system, liquid algae (cells suspended in solution) are placed on the surface of the dryer's conveyor belt. The belt is a food grade mylar (transparent polyester film) set on the surface of hot water. Heat from the circulating water is conducted to the belt and then into the water present in the product to be dried, gently speeding the natural process of evaporation while protecting natural nutrients. As the product dries and water evaporates, heat ceases to be transmitted to the product. Without being bound by theory, this prevents the degradation of polypeptides, nucleic acids, nutrients and pigments. Thus, the drying process maintains algae temperature far below the temperature of the circulating water beneath the conveyor belt.

Other drying systems can be used to produce dried algae, such as spray drying or freeze drying. Generally, two factors play a role in the degradation of algae: degree of heat and exposure time to heat. Applying a high amount of heat for a short period of time results in less degradation of the components of the blue-green algae. In one example, heat, such as a temperature of about 65° C. to about 80° C. is applied, such as a temperature of about 70° C. to about 75° C., or about 72° C. The heat can be applied for a sufficient amount of time to dry the algae, such as about 1 to about 15 minutes, or for about 2 to about 10 minutes, or for about 3 to about 7 minutes. In one example, heat is applied to the algae at 72° C. for only 3 to 5 minutes. This process is known to one of skill in the art, and is described in Abonyi et al., “Evaluation of Energy Efficiency and Quality Retention for the REFRACTANCE WINDOW™ Drying System: Research Report,” Washington State University, Pullman, Wash., Dec. 30, 1999). However, freeze dried cells can also be utilized.

As disclosed herein, an aqueous extract can be prepared from fresh, dehydrated, or preserved blue-green algae cells, such as AFA or Arthrospira. The algae can be extracted with water or a suitable buffered salt solution. For example, water or buffered solutions, general of a neutral pH (about pH 7.0 to about pH 7.8, such as about pH 7.2 to about pH 7.6, or about pH 7.4) is utilized. Suitable buffered salt solutions are well known in the art and include phosphate buffered saline (such as about 0.1 M phosphate buffered saline) and commercially available culture media. The aqueous extraction is generally performed below room temperature (generally 25° C.), such as at temperatures of about 3° C. to about 15° C., such as at about 4° C. to about 10° C., or at about 4° C., but the extraction can also be performed at room temperature (about 25° C.).

In one example, one gram of dried algal material, such as dried AFA or Arthrospira, is suspended in about 10 ml to about 50 ml, such as about 40 ml of water or phosphate-buffered saline (for example, 0.1 M phosphate buffered saline, pH 7.4), and incubated at 4° C. This incubation can last for 5 minutes, half an hour, several hours, or overnight. In several examples, the algae is incubated in an aqueous solution for about half an hour to about two hours, about half an hour to about three hours, or about half an hour to about 12 hours. The algae suspended in the water or buffered salt solution can be protected from light to decrease degradation. Following incubation in an aqueous solution, the solid material is separated from the aqueous extract. In some embodiments, the mixture of algae in the aqueous solution, such as the water or salt solution, can be mixed by repeated inversion of the vial, and centrifuged or filtered to remove solid material. For example, the suspension can be centrifuged at 4000×g for about 10 minutes, or filtered with an about 10 micron to about 0.22 micron filter, such as an about 1 micron to about 0.22 micron, or about 10 micron to about 0.45 micron filer

Following separation of the solid material, the supernatant, which generally appears blue in color, is isolated. This supernatant optionally can be sterilized, such as by filtration. In one example, a bright blue supernatant is decanted following centrifugation and sterile filtered using a 0.22 mm filter. This filtrate can be stored, such as at about 4° C. in the dark.

The extract can be dried, for example as described above. In one example, heat, such as a temperature of about 65° C. to about 80° C. is applied to the aqueous extract, such as a temperature of about 70° C. to about 75° C., or about 72° C. The heat can be applied for a sufficient amount of time to dry the extract, such as about 1 to about 15 minutes, or for about 2 to about 10 minutes, or for about 3 to about 7 minutes. In one example, heat is applied to the extract at 72° C. for only 3 to 5 minutes. This process is similar to the process for drying algae (see Abonyi et al., “Evaluation of Energy Efficiency and Quality Retention for the REFRACTANCE WINDOW™ Drying System: Research Report,” Washington State University, Pullman, Wash., Dec. 30, 1999). One of skill in the art can readily produce a dried product from an aqueous extract using known methodologies.

In several specific non-limiting examples, an effective amount of the composition including the mixture of AFA and Arthrospira aqueous extracts includes from about 0.25 grams to about 5 grams (such as from about 0.5 grams to about 5 grams, from about 0.5 grams to about 1 gram, from about 1 gram to about 1.5 grams, from about 1.5 grams to about 2 grams or from about 1 gram to about 2 grams of total extract.

In one specific non-limiting example, the effective amount of the composition including the mixture of AFA and Arthrospira aqueous extracts is about 0.1 grams to about 1.5 grams (such as about 0.1, about 0.2, about 0.3, about 0.4, about 0.5, about 0.75, about 1.0, about 1.25, or about 1.5 grams) of the mixture of dried aqueous extracts. In more embodiments, the effective amount of the composition including the mixture of AFA and Arthrospira aqueous extracts is about 0.1 grams to about 1.5 grams (such as about 0.1 to about 0.5 grams, about 0.5 to about 1.0 grams, about 10 to about 1.5 grams, about 0.1 to about 0.3 grams, about 0.3 to about 0.6 grams, about 0.6 to about 0.9 grams, about 1.0 to about 1.25 grams, about 0.9 to about 1.1 grams, about 0.4 to about 0.6 grams, about 0.1 to about 0.2 grams, about 0.2 to about 0.3 grams, about 0.3 to about 0.4 grams, or about 0.4 to about 0.5 grams)

The extracts and compositions disclosed herein can be administered in any form, including as solids such as tablets or powders or as a liquid preparation. In one example, the compositions are formulated for enteral administration. An example of a formulation of use is a pharmaceutical preparation (such as a tablet, enteral liquid, parenteral liquid, capsule, intranasal liquid or other form). In a particular disclosed example the composition is a pharmaceutical preparation, in particular a tablet or capsule. As is known in the art, compositions suitable for oral administration may be presented as discrete units or unit dosage forms such as capsules, cachets, or tablets, each containing a therapeutically effective amount of the composition, as a powder or granules, or as a solution or a suspension in an aqueous liquid. Thus, dosage forms include tablets, capsules, dispersions, suspensions, solutions, capsules and the like. Because of their ease of administration, tablets and capsules represent a convenient oral dosage unit form, in which case solid pharmaceutical carriers as described above are employed. However, the compounds can also be administered by controlled release means, or can be formulated for other means of delivery, such as, but not limited to intranasal or transdermal delivery.

The compositions can include inactive ingredients such as binding agents (such as pregelatinized maize starch, polyvinylpyrrolidone or hydroxypropyl methylcellulose); binders or fillers (such as lactose, pentosan, microcrystalline cellulose or calcium hydrogen phosphate); lubricants (such as magnesium stearate, talc or silica); disintegrants (such as potato starch or sodium starch glycolate); or wetting agents (such as sodium lauryl sulphate).

In one example, a tablet containing the compositions disclosed herein, can be prepared by compression or molding, optionally, with one more accessory ingredients. Compressed tablets can be prepared by compressing in a suitable machine, a free-flowing form such as powder or granules of a dried extract optionally mixed with a binder, lubricant, inert diluent, surface active or dispersing agent. The composition, such as the tablet, can include pharmaceutically acceptable components such as lactose, glucose, sucrose, corn starch, potato starch, cellulose esters such as cellulose acetate, ethyl cellulose, magnesium stearate, calcium silicate, precipitated silica, talc, fatty acids such as stearic acid, microcrystalline cellulose, carnauba wax and the like. The tablets or capsules can be coated by methods well known in the art.

Liquid preparations for oral administration can take the form of, for example, solutions, syrups or suspensions, or they can be presented as a dry product for constitution with water or other suitable vehicle before use (see the examples section). Such liquid preparations can be prepared by conventional means with pharmaceutically acceptable additives that are inactive agents, such as suspending agents (such as sorbitol syrup, cellulose derivatives or hydrogenated edible fats), emulsifying agents (such as lecithin or acacia), and preservatives (such as methyl or propyl-p-hydroxybenzoates or sorbic acid). The compositions can also be made to be pleasant tasting, and thus can contain buffer salts, flavoring, coloring and sweetening agents as appropriate.

Diluents and other inactive ingredients such as one or more pharmaceutically acceptable binding agents, fillers, supports, thickening agents, taste-improving agents, coloring agents, preservatives, stabilizers, regulators, emulsifiers, flow agents, absorbents, and the like or mixtures thereof may be used depending on the form of the composition employed. The composition can also include a sweetener, such as a natural (for example, sugar or honey) or artificial sweetener (for example, saccharine), if desired. Generally, the carriers, sugars, diluents, stabilizers, buffers, flavoring and texturing ingredients are considered to be inactive ingredients, as they do not impart a therapeutic effect in and of themselves.

In several embodiments, the composition can include one or more additional extract(s) of AFA or Arthrospira that can induce the migration of stem cells. The additional extract can be an alcohol extract, such as but not limited to, ethanol or methanol. In one example, the additional extract is produced by extracting AFA or Arthrospira in about 10% to about 20% ethanol. In one example, the composition includes an additional extract prepared by extracting liquid AFA or Arthrospira in about 10% ethanol. In one example, the additional extract is produced by incubating liquid AFA or Arthrospira in about 10% ethanol at a temperature of about 65° C. to about 85° C. is applied to the aqueous extract, such as a temperature of about 70° C. to about 95° C., or about 85° C. The solution is then centrifuged and the supernatant is dried (see above). In some embodiments, the composition can include a ratio of aqueous extract from AFA and aqueous extract from Arthrospira to the additional extract of 1:0.05, 1:0.1, 1:0.15, 1:0.2, 1:0.25, 1:0.3, 1:0.35, 1:0.4, 1:0.45, 1:0.5, 1:0.6, 1:0.7, 1:0.8, 1:0.9, 1:1, 1:1.1, 1:1.2, 1:1.25, 1:1.5, 1:1.75, or 1:2. In one embodiment, about 50 mg to about 500 mg, such as about 100 mg to about 250 mg, such as about 150 mg of the additional extract is included in the composition for administration to a subject.

In some embodiments, the composition includes the extracts of AFA and Arthrospira blended with dental type silica.

### Increasing Stem Cell Trafficking

A method is described herein for increasing stem cell trafficking (such as mobilization and/or homing) by administering to a subject a therapeutically effective amount of a composition including a mixture of an aqueous extract of AFA and an aqueous extract of Arthrospira, such as any of the compositions disclosed above. The subject can be any subject, such as a human or a veterinary subject.

The composition can be administered alone or in combination with other agents. In several embodiments, the mixture of the aqueous extract of AFA and the aqueous extract of Arthrospira (such as a solid form thereof) is included in a pharmaceutical composition along with a pharmaceutically acceptable carrier. Therapeutically effective amounts of additional components, such as solid forms of additional extracts, can also be administered to the subject. In one embodiment, a therapeutically effective amount of a solid form of mixture of the aqueous extract of AFA and the aqueous extract of Arthrospira is administered to the subject. Thus, a method is provided herein for increasing the trafficking of stem cells in a subject, comprising administering a therapeutically effective amount of a mixture of the aqueous extract of AFA and the aqueous extract of Arthrospira, thereby increasing the mobilization of stem cells in the subject.

In one specific, non-limiting example, the mixture of the aqueous extract of AFA and the aqueous extract of Arthrospira is dried, such that a solid form is produced, and a therapeutically effective amount of the solid form is administered to a subject of interest. In some embodiments, the therapeutically effective amount of the mixture of the aqueous extract of AFA and the aqueous extract of Arthrospira, can be from about 0.01 to about 1.0 g per kg body weight, such as about 0.05 to about 0.5 gram per kg body weight, or from about 0.1 to about 0.5 gram per kg body weight. In another specific, non-limiting, example the effective amount of the solid form of the mixture of the aqueous extract of AFA and the aqueous extract of Arthrospira can be from about 0.25 gram to about 5 gram, of from about 0.5 gram to about 5 gram, or from about 1 gram to about 2 gram. In one specific, non-limiting example, the effective amount of the solid form of the mixture of the aqueous extract of AFA and the aqueous extract of Arthrospira is about 1 gram. The active agents of the compositions disclosed herein can be admixed with a carrier. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some embodiments the auxiliary substances are non-naturally occurring substances.

This effective amount of the agent may be administered at a given frequency, such as about once a week, about twice a week, about three times a week, once a day, about twice a day, about three times a day, or more. One of skill in the art can readily determine a therapeutically effective amount of the mixture of the aqueous extract of AFA and the aqueous extract of Arthrospira. In one specific, non-limiting example, the amount of circulating stem cells, such as the amount of CD45−CD31+KDR+ stem cells, and/or the amount of CD34+ stem cells, can be assessed before and after administration of the composition.

The therapeutically effective amount of the mixture of the aqueous extract of AFA and the aqueous extract of Arthrospira, and the frequency of administration of these compositions, can depend on a variety of factors, such as the genus or species of algae utilized, the general health of the subject being treated, and the physiological characteristics (e.g., height, weight, body fat percentage, metabolism, etc.) of the subject being treated. In some embodiments, the mixture of the aqueous extract of AFA and the aqueous extract of Arthrospira is dried and can be administered as a solid. In another embodiment, the mixture of the aqueous extract of AFA and the aqueous extract of Arthrospira is dried, and then a specific amount can be dissolved in a carrier and subsequently administered to the subject.

Specific assays for determining a therapeutically effective amount of the mixture of the aqueous extract of AFA and the aqueous extract of Arthrospira. In one specific, non-limiting example, different amounts of the mixture of the aqueous extract of AFA and the aqueous extract of Arthrospira, are consumed by human subjects and the presence and/or quantity of stem cells (which can include subtypes of such cells) present in the circulatory system is detected and/or analyzed. In another embodiment, an animal (such as a mouse, rat, or other veterinary) model is utilized, and the population of newly integrated stem cells is monitored in various tissues (see the Examples below). It should be noted that the methods disclosed have equal application in medical and veterinary settings.

Regardless of how provided or administered, the mixture of the aqueous extract of AFA and the aqueous extract of Arthrospira can induce an increase in the population of circulating stem cells, such as CD34+ stem cells and/or CD45−CD31+KDR+ stem cells. The mixture of the aqueous extract of AFA and the aqueous extract of Arthrospira can also include an increase in stem cells that can be measured by fluorescently labeled aminoacetaldehyde. This procedure is described on the stem cell website (on line at stemcell.com/technical/aldefluor.asp and stemcell.com/technical/12_aldefluor.pdf, incorporated by reference herein in its entirety). Briefly, fluorescent-labeled aminoacetaldehyde can freely diffuse into cells. An intracellular enzyme ALDH (aldehyde dehydrogenase) converts this into fluorescent-labeled aminoacetate, which cannot diffuse out of the cells. Thus, cells that have the enzyme ALDH (such as stem cells) become fluorescent. Other cells (such as cells that are not stem cells, including differentiated cells) appear non-fluorescent after washing.

An increase in stem cell trafficking (such as an increase in circulating stem cells) may be measured by assaying the response of stem cells to a particular dose of the mixture of the aqueous extract of AFA and the aqueous extract of Arthrospira. In one embodiment, providing a mixture of the aqueous extract of AFA and the aqueous extract of Arthrospira to a subject can increase mobilization of that subject's stem cells within a certain time period, such as less than about 5 hours, less than about 4 hours, less than about 2 hours, less than about 1 hour, less than about 30 minutes, or less than about 10 minutes following administration.

In one embodiment, administration of a mixture of the aqueous extract of AFA and the aqueous extract of Arthrospira, results in the mobilization of stem cells into the circulation from about 10 to about 120 minutes following administration, such as from about 10 to about 30, about 30 to about 60, about 60 to about 120, or about 90 to about 120 minutes following administration. Mobilized stem cells will enter the circulatory system, thus increasing the number of circulating stem cells within the subject's body. The percentage increase in the number of circulating stem cells compared to a normal baseline may be about 25%, about 50%, about 100% or greater than about 100% increase as compared to a control. In one embodiment, the control is a baseline value from the same subject. In another embodiment, the control is the number of circulating stem cells in an untreated subject, or in a subject treated with a placebo or a pharmacological carrier.

In some embodiments, the subject is healthy. In other embodiments, the subject is suffering from a disease or physiological condition, such as immunosuppression, chronic illness, traumatic injury, or degenerative disease. In some embodiments, the extract composition is given to a subject to support the immune system, cardiovascular health, the health of bones and joints, the health of the brain and/or nerves, or improve liver function. In other embodiments, the composition is given to a subject to support recovery from injury, such as injury caused by trauma or disease.

Hematopoietic stem cells play a role in the continuous lifelong physiological replenishment of blood cells. Stem cells develop into both hematopoietic lineage cells and non-hematopoietic, tissue specific cells. Recently, stem cells have been found to differentiate into a variety of tissue-specific cell types, such as myocytes, hepatocytes, osteocytes, glial cells, and neurons. For example, stem cells have been shown to cross the blood-brain barrier (Willams and Hickey, Curr. Top. Microbiol. Immunol. 202:221-245, 1995) and differentiate into neurons (Mezey, Science 290:1779-82, 2000). Thus, it is possible that stem cells could be used to treat Parkinson's disease (Polli, Haematologica 85:1009-10, 2000), Alzheimer's disease (Mattson, Exp. Gerontol. 35:489-502, 2000), and traumatic brain injury (Magavi, Nature 405: 892-3, 895, 2000). Stem cells also have been shown to differentiate into fibroblasts or fibroblast-like cells, and to express collagen (Periera et al., Proc. Natl. Acad. Sci. 95:1142-7, 1998). Thus, it is possible that stem cells can be used to treat osteogenesis imperfecta and bone fractures. Peterson et al. (Science 284:1168-70, 1999) also has shown that liver cells can arise from stem cells. Thus, stem cells may be of use in treating a variety of pathologies of the liver, including, but not limited to cirrhosis. In addition, bone marrow derived stem cells have been demonstrated to migrate to the site of a myocardial infarction and form myocardium (Orlic, Nature 410:701-5, 2000). Thus, stem cells may be use in treating myocardial infarction.

Since stem cells are capable of differentiating into a broad variety of cell types, they play an important role in the healing and regenerative processes of various tissues and organs (see Koc et al., Bone Marrow Transplant, 27(3):235-39, 2001). Indeed, many studies suggest that the mobilization, migration and differentiation of bone marrow stem cells in the target tissue constitute a natural phenomenon of healing in the human body (Spencer et al., Thorax 60(1):60-2, 2005; Ishikawa et al., FASEB J. 18(15):1958-60, 2004; Mattsson et al., Transplantation 15; 78(1):154-7, 2004; Thiele J et al., Transplantation 77(12):1902-5, 2004; Cogle et al., The Lancet 363(9419):1432-7, 2004; Deb et al., Circulation 107(9):1247-9, 2003; Korbling et al., N Engl J Med 346(10):738-46, 2002; Adams et al., Blood 102(10):3845-7, 2002; Krause et al., Cell 105(3):369-77, 2001).

Accordingly, the disclosed methods of increasing mobilization and homing of stem cells in a subject can be used to treat or improve certain diseases or conditions. For example, in some embodiments, the subject suffers a disease or condition of the skin, digestive system, nervous system, lymph system, cardiovascular system, or endocrine system. In one example, the subject has a cardiovascular system disorder, such as atherosclerosis, myocardial infarction, arrhythmia, heart failure, a congenital heart defect, or cardiomyopathy.

### EXAMPLES

The following examples are provided to illustrate particular features of various described embodiments. The scope of the present invention should not be limited to those features exemplified.

### Example 1

### Production of AFA-w and SPIR-w

AFA was isolated from Klamath Lake. The AFA was dried using REFRACTANCE WINDOW™ Technology. Dried powder of Arthrospira platensis was obtained from Healthforce Nutritionals Inc, Escondido CA.

To generate aqueous extract of AFA (AFA-w) or aqueous extract of Arthrospira (SPIR-w), one gram of dried algal material (e.g., one gram of dried AFA, one gram of dried Arthrospira, or half a gram each of dried AFA and Arthrospira) was resuspended in 10 ml phosphate-buffered saline or water and incubated for one hour at 4° C. and protected from light. This slush was mixed by repeated inversion of the vial, and centrifuged at 400 g for 10 minutes. The bright blue supernatant was decanted and sterile filtered using a 0.22 mm filter. This filtrate was stored cold and dark, and used within the same day of preparation.

### Example 2

### Cellular Activation and Production of GM-CSF

This example illustrates that aqueous extracts of AFA and Arthrospira can be used to increase activation of stem cells through the production of stem cell related growth factors by other cell types.

### Activation of NKT Cells

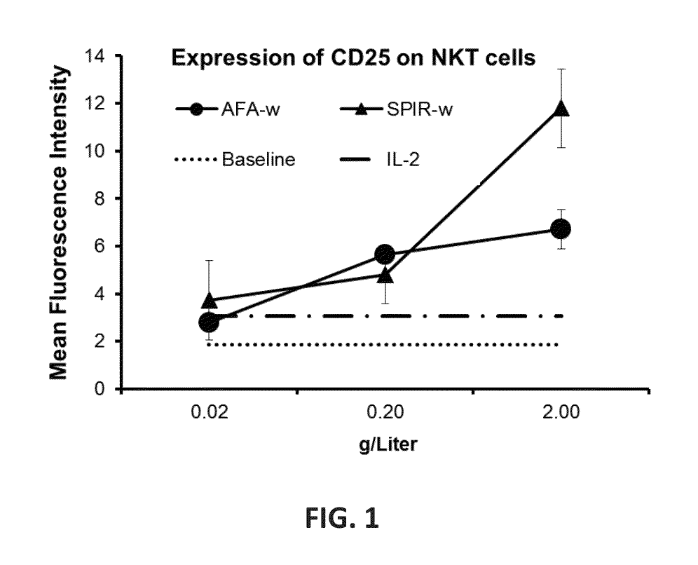

Natural killer T (NKT) cells when activated can secrete stem cell related growth factors such as GM-CSF. Both AFA-w and SPIR-w activated NKT cells to express the CD25 activation marker, which is associated with proliferation. As illustrated in FIG. 1, the induction of CD25 expression on NKT cells by AFA-w and SPIR-w was dose-dependent.

### Cellular Production of GM-CSF

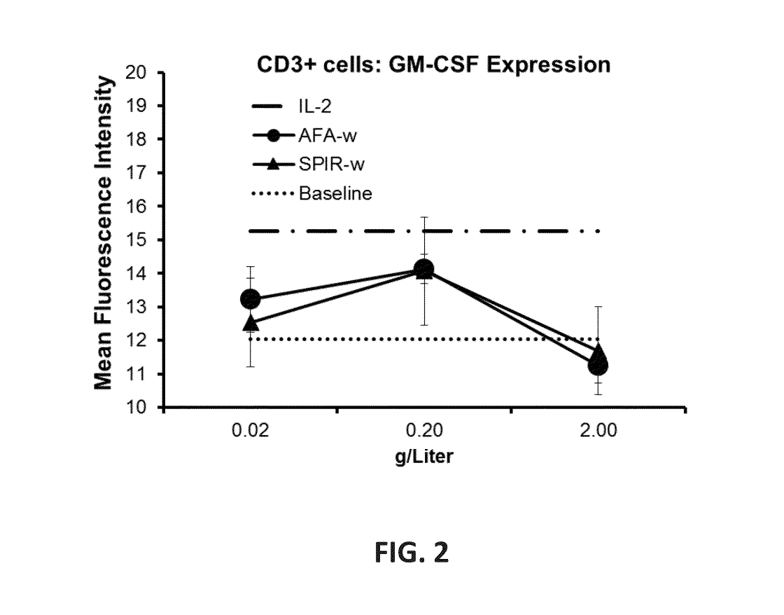

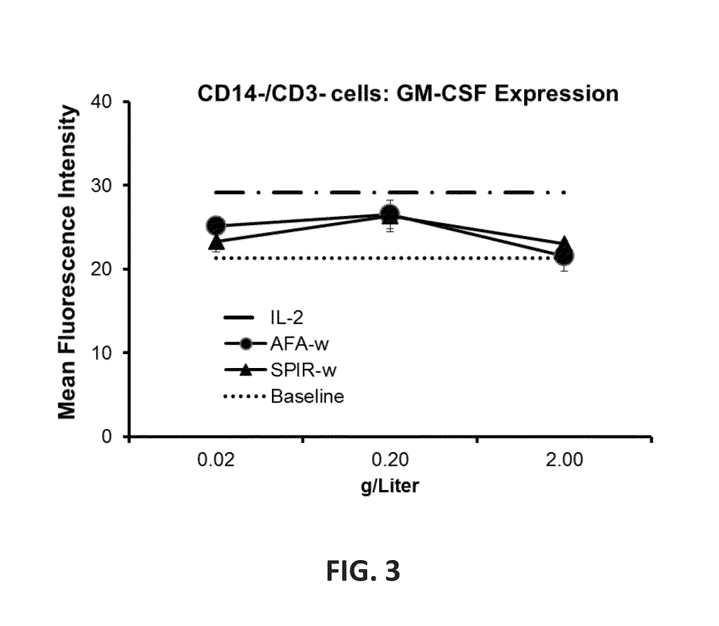

To further analyze the cellular effects of AFA-w and SPIR-w, CD3+ cells PBMCs and CD14−/CD3− PBMCs were incubated with increased amounts of AFA-W and SPIR-w, and expression of GM-CSF assayed by FACS analysis (FIGS. 2 and 3). The data on intracellular GM-CSF production in PBMC subpopulations show selective induction of GM-CSF production above baseline in both the CD14−/CD3− cell population, and even more noteworthy, in the CD3+ population which contains the NKT cell subset. NKT cells are known to be rapidly induced to produce GM-CSF (but not G-CSF), which suggests that NKT cells in the CD3+ subpopulation are responsible for the increased GM-CSF production. The induction of GM-CSF expression by AFA-w and SPIR-w in the CD14−/CD3− and the CD3+ cell population was comparable.

### Example 4

Increased CXCR4 chemokine receptor expression on two types of stem cells

This example illustrates that treatment of stem cells with a mixture of AFA-w and SPIR-w produces a synergistic increase in CXCR4 expression of the surface of stem cells compared to AFA-w or SPIR-w alone.

### Immunostaining for CXCR4 Expression on the CD34+Progenitor Cell Line KG1a

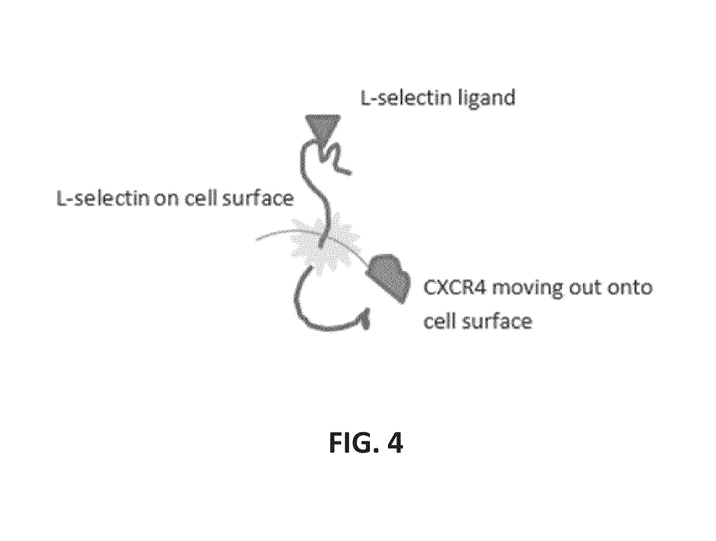

When the homing molecule L-selectin on the cell surface engages in ligand-binding, an intracellular signal is transmitted that leads to rapid and transient externalization of pre-made chemokine receptor CXCR4 (Duchesneau et al., Eur J Immunol. 2007 October; 37(10):2949-60; see FIG. 4). Externalization of CXCR4 makes the cell temporarily highly sensitive to recruitment signals from tissue, leading to increased “homing” of the cell to tissue. In the case of a circulating stem cell, this homing may be in response to injury and a need for repair.

This is followed by internalization, creating a window of time for responsiveness to chemotactic factors. Pretreatment of cells with AFA-w is known to reduce CXCR4 expression induced by the L-selectin ligand Fucoidan, and that AFA-w can induce a very mild and transient increase in CXCR4 expression (Jensen et al., Cardiovascular Revascularization Medicine 8 2007; 8:189-202).

A comparison of AFA-w, SPIR-w, and a 50:50 mixture of these extracts was tested for induction of CXCR4 from the KG1a cell line, which is a stem cell-like CD34+ progenitor cell line. The methods used were substantially as previously reported for determining CXCR4 induction (see Jensen et al., Cardiovascular Revascularization Medicine 8 2007; 8:189-202). To do so, cells from the stem cell-like CD34+ progenitor cell line KG1a were resuspended in RPMI at 10⁶ cells per milliliter and distributed in a series of round-bottom microwells. AFA-w was added to one series of wells, SPIR-w to another series, and a mixture of AFA-w and SPIR-w to the third series of wells. A dose comparison was included for AFA-w and SPIR-w.

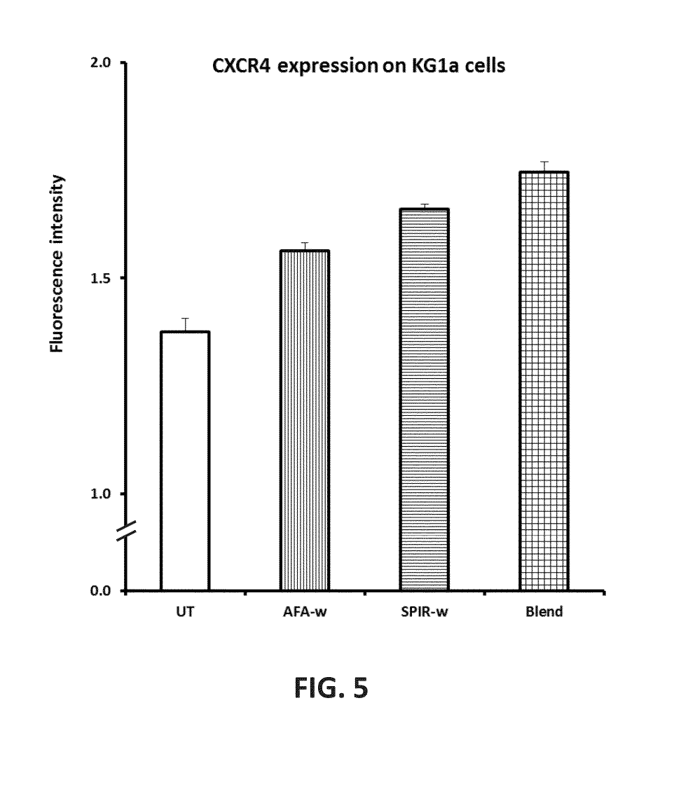

At different time points, saline containing sodium azide was added to wells in order to stop cytoskeletal movements and thereby stop the recycling of CXCR4 in and out of the cell. This allowed for examination of CXCR4 expressed at the cell surface at each time point. Cells were washed in saline containing sodium azide, stained with CXCR4-PE, fixed, and acquired by flow cytometry using an Attune acoustic flow cytometer. Analysis was performed by gating on viable cells using the forward and side scatter properties, then analyzing the KG1a cells for their CXCR4 mean fluorescence intensity, which is proportional to their CXCR4 expression. Surprisingly, although the mixture contained half the amount of AFA-w and SPIR-w as either of the extract alone conditions, an increase in CXCR4 expression was observed in the Blend condition compared to treatment with either of the extracts alone (FIG. 5). This finding indicates that the mixture of the aqueous extract of AFA and the aqueous extract of Arthrospira acts synergistically to increase CXCR4 expression on the cell surface of stem cells. As increased CXCR4 expression correlates with increased mobilization of stem cells from bone marrow to circulating blood and homing to target tissue, it is expected that the mixture would also increase mobilization and homing of cells in a subject.

### Example 5

### Human Trials

This example illustrated clinical testing to evaluate the effects of the mixture of AFA-w and SPIR-w on mobilization of adult stem cells.

The following products were tested*:

The following products were tested*:

| | Dose | Delivery |
|---|---|---|
| AFA-w | 950 mg | Veggie caps |
| SPIR-w | 950 mg | Veggie caps |
| AFA-w:SPIR-w 50:50 | 475 mgAFA-w:475 mgSPIR-w | Veggie caps |
| Placebo** | 1 capsule | Veggie caps |

*All products and placebo were encapsulated in rapidly dissolving veggie caps.
**Placebo: Rice flour, color-matched to the algae extracts, and encapsulated at NIS Labs.

This clinical testing compared the effects in vivo in 4 healthy volunteers (2 males, 2 females, age 45-59 years), after consumption of placebo or one of the test products on each study day, by measuring circulating stem cell populations before and at one hour and two hours after consumption.

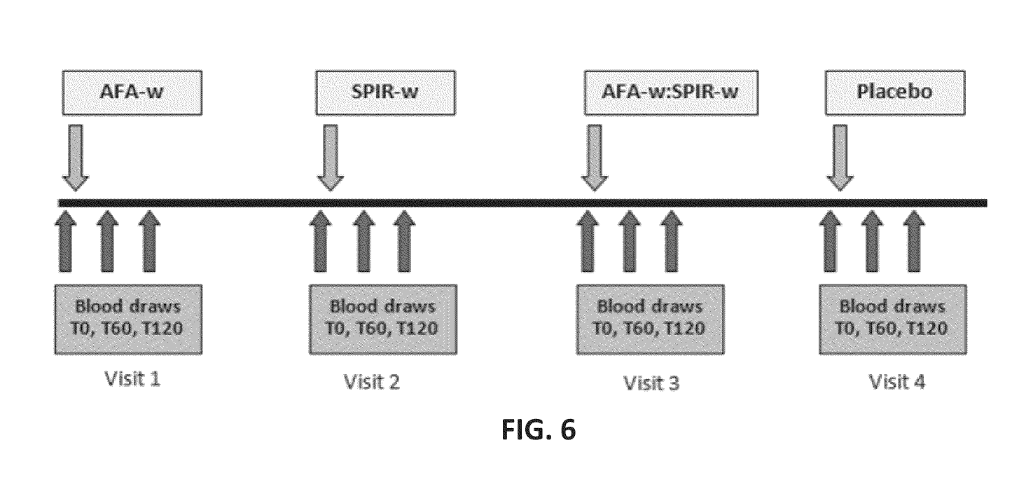

Upon arrival on the morning of each clinic day, participants rested quietly for one hour prior to baseline blood draw. Following the baseline blood draw a dose of product was fed to the participants, who did not know if the capsule included placebo or an active product. Two further blood samples were drawn at one and two hours after consumption. The volunteers were tested on separate clinic days with 7 days wash-out period between clinic days. The order in which the 4 products were consumed by study participants was randomized (see FIG. 6 for a schematic diagram of the study protocol).

The study participants were recruited according to the following inclusion/exclusion criteria:

### Inclusion Criteria:

1. Healthy adults 20-75 years of age;
2. BMI below 35;
3. Veins easy to see in both arms.

### Exclusion Criteria

1. Previous major gastrointestinal surgery (absorption of test product may be altered) (minor surgery not a problem);
2. Taking daily OTC medications (NSAIDS, allergy medications, and others) (birth control not a problem);
3. Taking anti-depressants or hypnotics;
4. Currently experiencing intense stressful events and life changes;
5. Actively depressed;
6. Experiencing sleep disturbances;
7. Working night shift;
8. Pregnant, nursing, or trying to become pregnant;
9. Food intolerances or allergies currently causing discomfort (such as Celiac's disease), due to ongoing inflammatory reactions that may negatively affect product absorption within the 3 hours of testing;
10. Food allergies related to ingredients in test product.

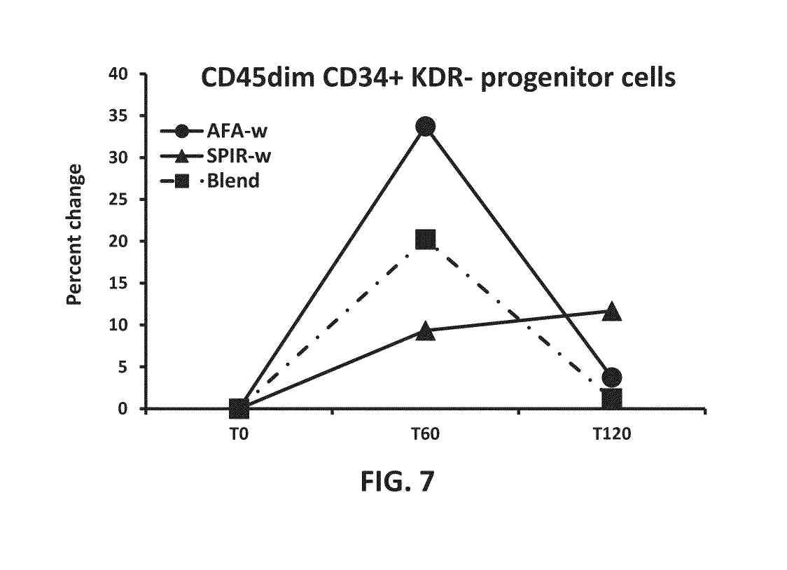

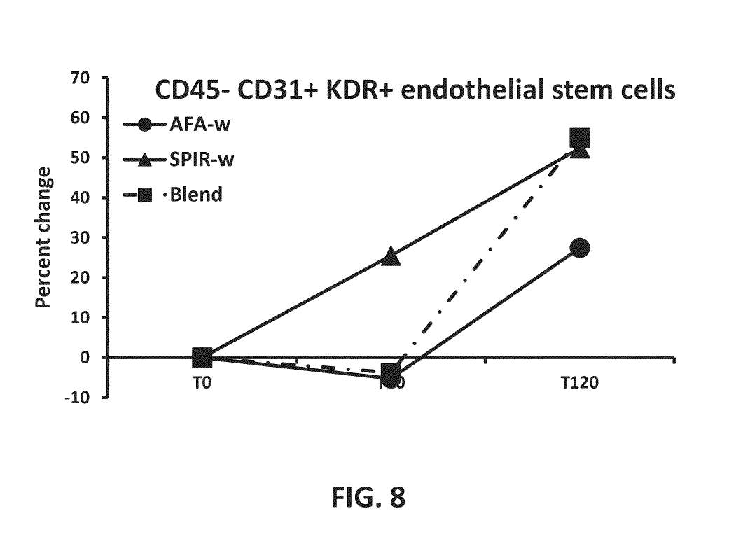

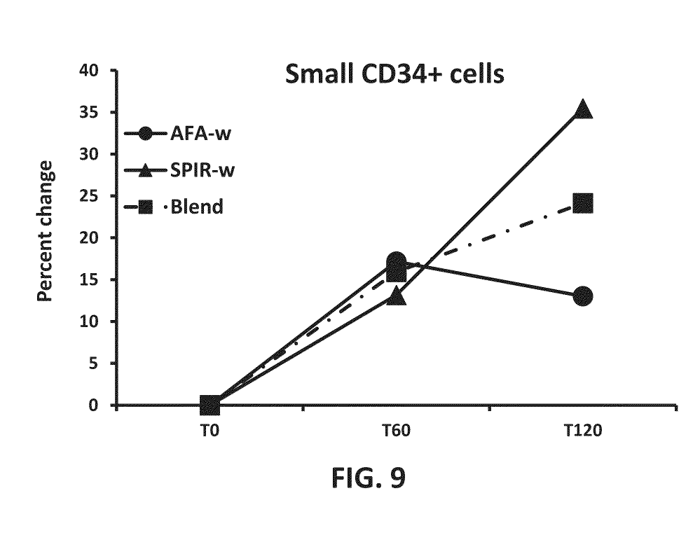

The blood samples were processed for immunophenotyping using simultaneous staining with CD31, CD34, CD45, and CD309 (KDR). Samples were acquired on an Attune acoustic dual-laser flow cytometer, and the data analysis performed to collect data on the numbers of different types of stem cells per microliter whole blood. The level (cells/μL) of each stem cell type was calculated for the placebo day and for each day involving a test product, for each study participant. The relative change for each study participant between placebo and test products were calculated, and these percent changes were averaged for the 4 people. FIGS. 7-9 show the data for the following stem cell types:
a) CD45dim CD34+KDR− progenitor stem cells (FIG. 7);
b) CD45− CD31+ KDR+ endothelial stem cells (FIG. 8);
c) Small CD34+ stem cells (FIG. 9).

As shown in FIG. 7, consumption of AFA-w provokes a rapid mobilization of CD45dim CD34+KDR− progenitor cells. Consumption of SPIR-w induces a slower release of this cell type, and the mixture produces an intermediate response.

Unexpectedly, CD45− CD31+ KDR+ endothelial stem cells showed about the same level of mobilization with the AFA-w/SPIR-w mixture as with SPIR-w alone (FIG. 8). This is surprising as it would be expected that treatment with the mixture would produce a mobilization level in between the AFA-w and SPIR-w treatments, because the mixture contains half the amount of AFA-w and SPIR-w. Interestingly, the effect was seen as the two hour post-administration time point, but not the one-hour post administration time point.

As shown in FIG. 9, Consumption of SPIR-w provokes a more robust mobilization of the very small CD34+ stem cells than AFA-w. The mixture provoked a level of mobilization between the AFA-w and SPIR-w.

### Example 6

### Stem Cells from Bone Marrow Populate Multiple Distant Tissues

A murine model can be used to evaluate the ability of stem cells mobilized by consumption of blue-green algae to populate distant tissues of the body. Male mice are selected as bone marrow donor animals, while all recipient mice are females. Female recipients are sub-lethally irradiated prior to injection of male bone marrow cells into their tail veins. Two groups of mice are evaluated. The first group of 20 animals are sub-lethally irradiated, injected with bone marrow, and put on normal feed. The second group of 20 animals is also sub-lethally irradiated, receive male bone marrow, and are fed a diet of normal feed plus 0.5 to 15% w/v of a mixture (such as a 50:50 mixture) of an aqueous extract of AFA and an aqueous extract of Arthrospira.

About 6×10⁶ nucleated cells of adult bone marrow is harvested from male mice aged 8-10 weeks and injected into the tail veins of sub-lethally irradiated isogenic adult female recipients, also aged 8-10 weeks. Mice from each group are sacrificed at each of the following time points: time 0, 1 week, 2 weeks, 3 weeks, 4 weeks, and 8 weeks. At time points 2 and 8 weeks, 6 mice are sacrificed from each group. At all other time points, 2 mice are sacrificed from each group.

During the first two weeks after injection, 15 microliters of whole blood is taken from the ear, tail, or paw, and immediately diluted in 200 microliters of buffer (phosphate buffered saline, pH=7.2, 2% serum, 0.02% azide) to dilute clotting factors and prevent coagulation. The blood samples are assayed to monitor the repopulation of platelets, red blood cells, and leukocytes within the blood. A portion of the blood sample is used for obtaining a cell count and for differential evaluation of red blood cells versus white blood cells. The sample is assayed using a flow cytometer, and the proportion of neutrophils, lymphocytes, and monocytes will be evaluated using forward and side scatter. The blood leukocytes will be examined for male origin using flow cytometry.

At time of sacrifice, various cell and tissue types will be examined for Hy antigen, which demonstrates that the cell or tissue originated in a male mouse. Brains are harvested and the entire brain is examined, including the olfactory bulb, hippocampus, cortical areas, and cerebellum. Bone marrow, heart muscle, hind leg muscle, liver, pancreas, sections of small intestine, and lung tissue are examined for presence of cells with Y chromosome, either by detection of surface Hy antigen by immunofluorescence, or by fluorescence in situ hybridization using probes for the Y chromosome. These data will document to what extent a diet containing blue-green algae promotes the homing, implantation, and differentiation process of the injected bone marrow stem cells.

### Example 7

### Increased Stem Cell Repopulation of Traumatized Tissue

A mouse model is used to evaluate homing and integration of bone marrow derived stem cells into traumatized tissue

All marrow donors are adult male mice (8-10 weeks of age), and all recipient mice are adult females (8-10 weeks of age). Two groups of mice are evaluated. One group of sub-lethally irradiated recipients receive 6×10⁶ nucleated donor cells via injection in the tail vein and allowed 2 weeks of recovery. The animals are then lightly traumatized by thin needle insertion into hind leg muscle, heart, and brain. All animals receive normal feed throughout the study. In the second group, female mice are treated identically as the first group, but are fed a diet that includes 0.5 to 15% w/v of a mixture (such as a 50:50 mixture) of an aqueous extract of AFA and an aqueous extract of Arthrospira.

Two mice are sacrificed prior to trauma to evaluate baseline levels of male-derived cells. Subsequently, mice are sacrificed at the following time points: 1 week, 2 weeks, 3 weeks, and 4 weeks. Two mice are sacrificed for each time point, except for the 2 week time point, where 6 mice are sacrificed from each group. Hind leg muscle, heart, and brain tissue is isolated from the sacrificed animals. Sections are cut through the traumatized areas, and stained for male-derived cells using either cell surface marker analysis for the expression of the Hy antigen or by fluorescence in situ hybridization using probes for the Y chromosome.

Data obtained demonstrate the effect of consuming the mixture of the aqueous extract of AFA and the aqueous extract of Arthrospira on the speed of stem cell recruitment following trauma.

It will be apparent that the precise details of the methods or compositions described may be varied or modified without departing from the spirit of the described invention. We claim all such modifications and variations that fall within the scope and spirit of the claims below.

It is claimed:

1. A composition, comprising a first component and a second component, wherein:
the first component comprises an aqueous extract of Arthrospira; and
the second component comprises an aqueous extract of Aphanizomenon flos aquae.

2. The composition of claim 1, wherein
(a) the aqueous extract of Arthrospira comprises an aqueous extract of fresh, dehydrated, or preserved Arthrospira;
(b) the aqueous extract of Aphanizomenon flos aquae comprises an aqueous extract of fresh, dehydrated, or preserved Aphanizomenon flos aquae; or
(c) both (a) and (b).

3. The composition of claim 1, wherein
(a) the first component consists of a dried form of the aqueous extract Arthrospira; and
(b) the second component consists of a dried form of the aqueous extract of fresh, dehydrated, or preserved Aphanizomenon flos aquae; or
(c) both (a) and (b).

4. The composition of claim 1, wherein the first component and the second component are mixed at a ratio of about 10:90 w/w to about 90:10 w/w.

5. The composition of claim 1, wherein the first component and the second component are mixed at a ratio selected from the group consisting of about 5:95 w/w, about 10:90 w/w, about 20:80 w/w, about 30:70 w/w, about 40:60 w/w, about 50:50 w/w, about 60:40 w/w, about 70:30 w/w, about 80:20 w/w, about 90:10 w/w, and about 95:5 w/w.

6. The composition of claim 1, wherein the Arthrospira comprises Arthrospira platensis, Arthrospira maxima, or both.

7. The composition of claim 1, wherein the aqueous extract is a water extract or a buffered saline extract.

8. The composition of claim 1, comprising about 0.5 grams to about 5 grams of the mixture of the first and second components.

9. The composition of claim 8, where in the composition comprises about 0.5 grams of the first component and about 0.5 grams of the second component.

10. The composition of claim 1, wherein the composition is a solid.

11. The composition of claim 10, wherein the solid is encapsulated.

12. A liquid composition, comprising the composition of claim 1 dissolved in water.

13. A composition comprising a therapeutically effective amount of the composition of claim 1 in a pharmacologically acceptable carrier.

14. A method of increasing stem cell trafficking in a subject, comprising:
administering to the subject a therapeutically effective amount of the composition of claim 1, thereby increasing stem cell trafficking in the subject.

15. The method of claim 14, wherein the subject is a mammal.

16. The method of claim 15, wherein the subject is a human.

17. The method of claim 14, wherein increasing stem cell trafficking comprises an increase in the number of circulating stem cells in the subject.

18. The method of claim 14, wherein the stem cell is an endothelial stem cell.

19. The method of claim 18, wherein the stem cell is a CD45−CD31+KDR+ stem cell.

20. The method of claim 19, further comprising measuring CD45−CD31+KDR+ stem cells in the subject.

21. The method of claim 14, wherein the stem cell is a hematopoietic stem cell.

22. The method of claim 21, wherein the stem cell is a CD34+ stem cell.

23. The method of claim 22, further comprising measuring CD34+ stem cells in the subject.

24. The method of claim 14, wherein increasing stem cell trafficking comprises an increase in stem cell mobilization and/or homing of about 50% to about 500% in the subject as compared to a control.

25. The method of claim 14, wherein the subject is a healthy subject, or is a subject with a chronic illness, traumatic injury, osteoporosis, Alzheimer's disease, cardiac infarction, Parkinson's disease, traumatic brain injury, multiple sclerosis, cirrhosis of the liver, a digestive system disorder, a nervous system disorder, a lymph system disorder, cardiovascular system disorder, an endocrine system disorder, a degenerative disease, or is immunosuppressed.
